(19) United States
(12) Patent Application Publication
(10) Pub. No.: US 2017/0042936 A1
(43) Pub. Date: Feb. 16, 2017

(54) COMPOSITION FOR LOCAL AND CONTROLLED RELEASE OF DRUGS AND METHODS THEREOF

(71) Applicants: INEB - INSTITUTO NACIONAL DE ENGENHARIA BIOMÉDICA, Porto (PT); ISEP - INSTITUTO SUPERIOR DE ENGENHARIA DO PORTO, Porto (PT)

(72) Inventors: Fernando Jorge MONTEIRO, Porto (PT); Susana Maria RIBEIRO E SOUSA MENDES DE FREITAS, Porto (PT); Catarina COELHO, Porto (PT); Nuno Paulo ALEGRETE DA SILVA, Porto (PT)

(21) Appl. No.: 15/305,435

(22) PCT Filed: Apr. 21, 2015
(86) PCT No.: PCT/IB2015/052916

§ 371 Date: Oct. 20, 2016

(30) Foreign Application Priority Data
Apr. 21, 2014 (PT) 107597

(51) Int. Cl. A61K 33/42; A61K 45/06; A61K 9/00; A61K 9/50

(52) U.S. Cl. CPC A61K 33/42; A61K 9/5052; A61K 9/5036; A61K 45/06; A61K 9/0019; A61K 9/5089

(57) ABSTRACT

The present disclosure relates to a composition for treatment and/or prevention of infections, namely bone diseases, in particular osteomyelitis, via a controlled release of antibiotics and subsequently induce regeneration of bone tissue that often undergoes necrosis due to infection. The present disclosure relates in particular to a pharmaceutical composition comprising one or more granules containing calcium phosphate, collagen and one or more polymers of heparin, and an antibiotic in an effective therapeutic amount, wherein the antibiotic is bound to the heparin polymers.

### TECHNICAL FIELD

The present disclosure relates to a composition for treatment and/or prevention of infections, namely bone diseases, in particular osteomyelitis, via a controlled release of antibiotics and subsequently induce regeneration of bone tissue that often undergoes necrosis due to infection.

### BACKGROUND

Osteomyelitis is a bone infection that may be caused by a variety of microorganisms, being the bacteria Staphylococcus (S.) aureus the pathogen most often found. The progress of this infection leads to necrosis of bone tissue. The current treatment of this infection involves debridement of the infected tissue and/or prolonged administration of an antibiotic to eradicate bacteria

Despite advances in the treatment of osteomyelitis, is still very difficult for orthopaedic doctors to control this infection, particularly when caused by resistant strains of S. aureus. Orthopaedic implants are medical devices very susceptible to infections, which are mainly caused by S. aureus or coagulase-negative staphylococci (e.g. S. epidermidis). In many cases, the prosthesis must be removed and, where possible, replaced, which implies a significant impact in terms of morbidity, mortality and medical costs. The recommended treatment of osteomyelitis involves debridement of the infected tissue and/or prolonged administration and parenteral penicillin or vancomycin. However, such treatment requires a prolonged stay in hospital, which requires high economic costs. On the other hand, parenteral administration of antibiotics requires the use of intravenous catheters that have associated their own risks of infection.

The current treatment of osteomyelitis has several limitations. For the debridement of tissue, it is not always possible to remove all the infected tissue. Thus, some bacteria may be quiescent in place and arise new bone infected. Regarding the prolonged administration of antibiotics, this requires a prolonged stay in hospital as well as the use of intravenous catheters which themselves have their onset risk of infection. On the other hand, a prolonged administration of antibiotics, a worldwide concern, leads to the emergence of resistant bacterial strains and it is therefore increasingly difficult to fight the infection, as the effectiveness of treatment decreases, patients remain infectious for a longer time and increases the risk of spreading resistant microorganisms to others. The infection itself causes bone defects that hinder the penetration of the antibiotic at the site and, consequently, the elimination of bacteria.

The limitations above-mentioned elucidate the great difficulty of treating osteomyelitis and demonstrate the importance of developing a new therapeutic strategy for osteomyelitis.

### General Description

The present solution describes a composition/material comprising calcium phosphate, collagen and heparin capable of delivering compounds, in particular antibiotics, for the in situ treatment of bone tissue infections, and, simultaneously, the composition/material functions as an osteoinductive material able to fill bone cavities. This composition/material can be used:
to immobilize antimicrobial agents, in particular antibiotics or cationic peptides;
in the treatment of infections associated with bone tissue;
as a transport vehicle for anti-tumoral drugs for treating bone metastases;
to carry bone morphogenic proteins, for treatment in cases of delayed consolidation of bone fractures.

In particular, this composition/material is of great interest for the use in medicine, in particular for the treatment of bone/cartilage infections namely osteomyelitis and may be used with a dual action:
to release of an antibiotic in a sustained and controlled manner over time and therefore promoting the effectiveness for treatment of infections by organisms as S. aureus; in particular to release vancomycin, tobramycin, cefadroxil, cephamycin, cefazolin, cephalexin, cefaclor, cefoxitin, cefprozil, cefuroxime, cefdinir, cefixime, cefoperazone, cefotaxime, cefpodoxime, ceftazidime, ceftibuten, ceftozoxima, ceftriaxone, cefepime, rifampin, ofloxacin, fusidic acid, trimethoprim-sulfamethoxazole, penicillin or their mixtures, among others;
to promote bone regeneration after elimination of the infection. The composition/material may be applied by implantation, injection or other form in the bone defects during surgery, not requiring the removal of the composition/material insert, since after infection control the material is absorbed and integrated in the body to promote regeneration and additionally in a synergetic way also decrease the infection.

This composition/material of the present subject matter may be used for fractionation of proteins by selective adsorption, thus allowing their separation.

An aspect of the present invention is related to a pharmaceutical composition comprising
one or more granules containing calcium phosphate, collagen and one or more polymers of heparin, and
an antibiotic in an effective therapeutic amount,
wherein the antibiotic is bound to the heparin polymers.

This composition surprisingly increased the antibiotic residence time in situ allowing to decrease of infections and maintaining the clinical effect, thus improving the cost/effect ratio of the treatment of infections, namely bone diseases, in particular osteomyelitis improving the tissue regeneration.

In an embodiment, the composition of the present invention may comprise the calcium phosphate granules coated with collagen.

In an embodiment of the composition of the present disclosure the heparin polymers may be bound to the collagen.

In an embodiment of the composition of the present disclosure the antibiotic may be a glycopeptide or penicillin or mixtures thereof, preferably an antibiotic selected from the following list: vancomycin, tobramycin, cefadroxil, cephamycin, cefazolin, cephalexin, cefaclor, cefoxitin, cefprozil, cefuroxime, cefdinir, cefixime, cefoperazone, cefotaxi me, cefpodoxi me, ceftazidime, ceftibuten, ceftozoxi ma, ceftriaxone, cefepime, rifampin, ofloxacin, fusidic acid, trimethoprim-sulfamethoxazole, penicillin or their mixtures.

In an embodiment of the composition of the present disclosure
a 40-55% (w/v) nanohydroxiapatite slurry was used, preferably 52% (w/v), for the preparation of granules of calcium phosphate with
0.01-10% (w/v) collagen, preferably 0.05% (w/v);
0.5-20% (w/v) heparin, preferably 2% (w/v);
0.5-20% (w/v) of an antibiotic, preferably 2.5% (w/v).

In an embodiment of the composition of the present disclosure, the granules of calcium phosphate aggregates comprise dimensions between 0.01-10 mm, preferably 0.1-5 mm, more preferably between 0.5-1.5 mm.

In an embodiment of the composition of the present disclosure, the collagen may be type I collagen.

In an embodiment of the composition of the present disclosure, the calcium phosphate granules may be hydroxyapatite granules.

In an embodiment of the composition of the present disclosure, the heparin has a molecular weight between 17 000-19 000 Da.

In an embodiment of the composition of the present disclosure, the coated granules further comprise a crosslink agent, the crosslink agent is preferably selected from the following list: N-(3-dimethylaminopropyl)-N′-ethylcarbodiimide, N-hydroxysuccinimide, or their mixtures.

In an embodiment of the composition of the present disclosure, the granules of calcium phosphate aggregates crosslinked with type I collagen are further coated with heparin.

In an embodiment of the composition of the present disclosure, the composition can be an injectable form.

Another aspect of the present invention is the use of the composition of the present disclosure in human medicine or veterinary, namely the use of such composition in the regeneration of bone and/or cartilaginous tissues, or treatment/prevention of bone and/or cartilaginous infections; in particular the use of such composition in treatment/prevention of bone and/or cartilaginous infections, more in particular the use of such composition for prevention or treatment of osteomyelitis.

Another aspect of the present invention also relates with a biomaterial comprising the composition of the present disclosure, preferably the material is composed of granule of any shape with dimensions between 0.01-10 mm.

Another aspect of the present invention also relates with a method for obtaining the composition of the present disclosure or the biomaterial comprising the following steps:
impregnating at least one polyurethane sponge with a calcium phosphate slurry, in particular nanohydroxyapatite slurry, to obtain an impregnated sponges;
drying the impregnated sponges;
heat-treating of the impregnated sponges in a sintering furnace until the sponge evaporate in order to obtain calcium phosphate material, preferably nanohydroxyapatite material, breaking and sieving the obtained calcium phosphate material, in particular nanohydroxyapatite material, in granules with sizes between 0.8-2.0 mm, preferably 1.18-1.70 mm;
coating the granules, in particular by dipping or spraying, with a collagen solution; adding a crosslink agent to immobilize polymers of heparin on the granules surface;
adding a suitable antibiotic to the granules, in particular by dipping or spraying in an antibiotic solution.

In an embodiment, the drying step is perform at 37° C. for 30 min.

In an embodiment, the heat-treating step comprises:
a heating rate step of 1° C./min till 600° C. with 1 h plateau;
a heating rate step of 4° C./min till 830° C. with 1 h plateau;
natural cooling step inside the furnace.

In an embodiment, the crosslinking step is carried out for 2-10° C. for 30 min 4 h, more preferably at 4° C. for 2 h.

Nanohydroxyapatite (NanoHA) is a calcium phosphate that is very similar to the apatite in the bone matrix, since it presents a hierarchical structure with nanometer dimensions. Compared with the microstructured hydroxyapatite (microHA), the NanoHA has an improved performance due to its surface area/volume ratio, and higher surface reactivity. In fact, NanoHA has properties that can control the adsorption of proteins, such as grain size, pore and hydrophobicity. It is also known that NanoHA is capable of improving the behavior of osteoblasts such as proliferation, mineral deposition of calcium and synthesis of ALP. The nanoHA has been studied as a promising material for drug delivery, in particular of vancomycin.

Type I collagen is a natural polymer present in the bone matrix and has already been used in tissue engineering. This polymer has been tested to improve the mechanical properties of hydroxyapatite (HA) and its biological properties. In fact, the combination of these two materials, HA and type I collagen, is the most direct approach to get the true artificial bone material, since it has a composition/material and nanostructure biological response similar to bone.

Heparin is a glycosaminoglycan present in the extracellular matrix and is able to interact with significant affinity with several relevant biomolecules such as growth factors and other proteins. Thus, heparin immobilization at biomaterials may improve the performance of drug delivery systems providing a controlled release. In fact, this glycosaminoglycan has been immobilized in HA/collagen scaffolds and proved to be successful for controlled release of BMP-2 protein. Therefore, heparin immobilized on the surface of a material can lead to the creation of a controlled release system of a given biomolecule.

Vancomycin is a glycopeptide with a molecular weight of approximately 1450 Da and is very effective in combating infections caused by gram-positive bacteria, particularly S. aureus. Resistant S. aureus strains are more frequently found in cases of osteomyelitis, including strains resistant to methicillin (MRSA). In such cases, vancomycin is the most adequate antibiotic to be administered. This antibiotic has low cytotoxicity to human osteoblasts, which is an important factor for application in bone tissue.

Of all the structural possibilities that could use the HA, the porous granules are more advantageous in cases of bone defects or irregular cavities, such as those found in patients with osteomyelitis. Furthermore, porous structures allow diffusion of nutrients, cell migration and bone growth factors critical to tissue regeneration.

The solution of the present disclosure has a similar constitution of bone extracellular matrix as it comprises an organic phase collagen fibres, in particular type I collagen fibres, and an inorganic phase of nanosized crystals of calcium phosphate, in particular HA. These materials, in particular nanoHA and type I collagen fibres are available for interaction with cells, proteins or other macromolecules, thereby mimicking the bone environment. Furthermore, these materials (nanoHA and type I collagen fibres) are further heparinized thus having heparin immobilized at the surface and furthermore an antibiotic. The set of up to several hundreds of nanosized crystals of calcium phosphate (nanoHA) corresponds to what it will be called aggregates. The association of these aggregates in vast numbers into tridimensional constructs presenting interconnective macro, micro and nanoporosity, and that include, in particular type I collagen, heparin and antibiotic, corresponds to what it will be called granules or composition/material.

These granules were produced using the method of impregnation of polyurethane sponge. The use of this method allowed to obtain scaffolds that were then broken and sieved to give granules with sizes between 1.18 and 1.70 mm. Subsequently, the crosslinking of both collagen and heparin immobilization was performed using N-(3-dimethylarninopropyl)-N′-ethylcarbodiimide/N-hydroxysuccinimide (EDC/NHS) as crosslinking reagents. The EDC/NHS are considered non-toxic reagents and capable of catalysing the formation of covalent bonds between the polymeric chains and between collagen and heparin, enabling the immobilization of heparin.

In an embodiment, the irregular morphology (various sizes and shapes) of granules is an advantage of the present solution as it allows adapting to bone defects formed by bacteria in the case of osteomyelitis and is therefore more versatile than structures with a pre-defined size.

The composition/material now disclosed also has a design with different pore sizes (macro, micro and nanoporosity) that allows cell migration, growth of bone tissue, diffusion of nutrients, protein adsorption and cell adhesion. The heterogeneous distribution of the collagen allows access to both the organic and inorganic components and is therefore very similar to natural bone environment. It was also unexpectedly found that this composition/material is capable of releasing in a controlled manner an antibiotic, in particular vancomycin, over time. Thus, the composition/material of the present disclosure is responsible for releasing the drug in a controlled manner and with an appropriate therapeutic window. The bioactive drug is released and is therefore capable of inhibiting bacterial growth, eradicating bacteria locally and significantly improve the local treatment of bone diseases, in particular osteomyelitis.

Bacterial adherence assays were performed which surprisingly confirmed the greater interaction with S. aureus materials of the present solution containing collagen and heparin. Thus, the presence of collagen and heparin in the material also causes the bacteria to be attracted to the site where the antibiotic release is occurring being this factor contributing to more effective eradication. The composition/material is cytocompatible and suitable for accession and bone cell growth, for example osteoblasts. Moreover, it is also a composition/material with appropriate characteristics to induce the regeneration of bone tissue.

Due to the presence of heparin in the composition/material, which has negatively charged groups (carboxyl and sulphur), the composition/material has the potential as an affinity purification material, to separate biomolecules with affinity for heparin, as fibroblast growth factor (FGF), vascular endothelial growth factor (VEGF), fibronectin, gamma interferon, among others.

This composition/material thus has a diversified set of applications, such as orthopaedic, dental and maxillofacial surgery. It is also used in scientific research as a model for treating problems associated with bone tissue, such as metastases, infections and tumours in the bone. Even in scientific research, it may be used as a composition/material for proteins separation.

An ideal strategy would combine the use of a material similar to bone matrix and regenerative capacity, as well as being capable of releasing in a controlled manner antibiotic and eradicate the bacteria causing the infection. Composites consisting of collagen and calcium phosphate are suitable for bone tissue regeneration applications because they mimic the components of the extracellular matrix, particularly the organic portion, in particular collagen type I and the inorganic part, in particular calcium phosphate. As mentioned above, vancomycin is effective in the eradication of S. aureus cases including methicillin-resistant strains (methicillin-resistant S. aureus, MRSA). This antibiotic also has advantageous characteristics for applications in bone osteoblasts as low cytotoxicity to not significantly affect the number of cells and the alkaline phosphatase activity (ALP). On the other hand, this antibiotic has a low minimum inhibitory concentration (MIC), for S. aureus, which makes unnecessary the use of very high concentrations. A major challenge of drug delivery is to achieve an appropriate release profile. The appropriate release profile should allow a considerable amount of drug to be released in the beginning, but after maintaining a controlled concentration over time in order to eradicate the microorganisms effectively. Previous studies of vancomycin release from calcium phosphate substrates have been conducted, including from substrates made of hydroxyapatite. However, the release profiles obtained were not optimal, consisting of initial busts followed by immediate drops to very low values, hence the elution of the antibiotic was not adequately controlled over time, as required for eradication of bacteria.

Based on the above-mentioned requirements a novel composition/material with high potential for improving the treatment of osteomyelitis has been developed. This product comprises porous granules of nanohydroxyapatite (NanoHA) and type I collagen fibres distributed on the surface of NanoHA. Heparin was immobilized in this material, thus producing heparinized NanoHA/collagen granules and finally antibiotic adsorption to the granules was conducted.

The characterization of the composition now disclosed was performed by scanning electron microscopy (SEM) and computed X-ray microtomography (micro-CT) and revealed irregular morphology of the granules, the presence of collagen and macro, micro and nano-porosity. After vancomycin adsorption onto the granules, its releasing profile was studied by UV molecular absorption spectroscopy. The heparinized granules surprisingly presented a more sustainable release over time, in comparison with nonheparinized nanoHA and nanoHA/collagen granules. Vancomycin was released for 360 h (FIG. 4) and proved to be bioactive until 216 h (FIG. 5). Staphylococcus aureus adhesion was higher on granules containing collagen, guiding the bacteria to the material with antibiotic, improving their eradication. Moreover, cytotoxicity of the released vancomycin was assessed using osteoblast cultures, and after 14 days of culture in the presence of vancomycin, cells were able to remain viable, increasing their metabolic activity and colonizing the granules, as observed by scanning electron microscopy and confocal laser scanning microscopy.

Therefore, heparinized nanoHA/collagen granules are the ideal material to improve the treatment of osteomyelitis, as they are capable of releasing vancomycin, eliminating the bacteria, and presented morphological and chemical characteristics to induce bone regeneration.

The solution now disclosed relates to a composition that may be used for implantation, injection or any other method were it is necessary to have a controlled local release of an antibiotic, for a certain period of time, for the treatment and prevention of infections, in particular in bone. The composition now disclosed allows eradication of infections of the surrounding tissues and concurrently promoting the regeneration of bone tissue and/or cartilaginous, and removal of the material after application to the affected area is not necessary.

The composition/material comprises granules of nanocrystalline calcium phosphate aggregates covered with collagen and heparin and a sufficient and effective amount of antibiotic to produce bactericidal effects in tissues and, in particular in the host bone. Furthermore, the composition/material may be applied to fill a bone defect in any way.

The above-mentioned composition/material comprises granules having sizes that do not inhibit tissue regeneration, and instead promote tissue regeneration. Furthermore, the composition/material is also to be remained in the treatment zone. The size of the granules is between 0.01 mm to 10 mm, preferably 0.1 mm to 5 mm, more preferably between 0.5 mm and 1.5 mm.

A composition/material wherein the calcium phosphate aggregates is nanocrystalline, and the calcium phosphate granules obtained by any method that allows to obtain about 1 mm particle size.

A composition/material wherein the granules of calcium phosphate aggregates are partially coated with collagen, preferably type I collagen and with heparin. The collagen, in particular type I collagen, and the heparin solutions are prepared in non-denaturing conditions using non-toxic solvents.

A composition/material used for controlled delivery of an antibiotic wherein the antibiotic compound comprises 1% to 10% of the composition/material and may be selected from the following list: vancomycin, tobramycin, cefadroxil, cephamycin, cefazolin, cephalexin, cefaclor, cefoxitin, cefprozil, cefuroxime, cefdinir, cefixime, cefoperazone, cefotaxime, cefpodoxime, ceftazidime, ceftibuten, ceftozoxima, ceftriaxone, cefepime, penicillin, or their mixtures.

The present solution also discloses a method based on the use of the composition/material previously described for controlled release of antibiotics for the treatment and prevention of infections and/or regeneration of bone or cartilage tissue, for application in the body of humans or other mammal, in an infected area or of potential infection, allowing the composition/material to locally deliver an effective amount of the antibiotic sufficient to reach bactericidal levels in adjacent tissues and in which the antibiotic is delivered in a linear dose for a minimum period of 2 weeks at levels that exceed the minimum inhibitory concentration of the causative organisms infection, especially infection is osteomyelitis.

Method wherein the step of implantation, injection or other application method of referred granules into the body involves placing the granules in a surgical treatment area; in a bone fracture zone; in surgical zone implants, screws, plates and metallic fixtures and/or involves placing the granules in an infected area for the treatment of osteomyelitis.

### BRIEF DESCRIPTION OF THE DRAWINGS

The following figures provide preferred embodiments for illustrating the description and should not be seen as limiting the scope of solution.

FIG. 1: SEM images from nanoHA/collagen granules. A: Presence of interconnective macroporosity; BD: Collagen distribution in a fiber-like structure on nanoHA; E: Presence of microporosity; F: Presence of nanoporosity.

FIG. 2: 3D micro-CT images from a nanoHA/collagen granule from different perspectives, showing interconnective macroporosity throughout all the granules structure.

FIG. 3: A—ATR-FTIR spectra of crosslinked collagen, nanoHA, nanoHA/collagen, and heparinized nanoHA/collagen granules; B—Magnification between 1150 and 1800 cm⁻¹ for crosslinked collagen, nanoHA, nanoHA/collagen, and heparinized nanoHA/collagen granules, indicating the respective relevant peaks.

FIG. 4: Vancomycin release from nanoHA, nanoHA/collagen, and heparinized nanoHA/collagen granules versus time. The values correspond to the concentration present at each time point. *Represents a statistically significant difference compared with nanoHA and nanoHA/collagen for each time point (p<0.05).

FIG. 5: A—Total number of S. aureus in the absence of vancomycin, for 0 and 24 h of incubation, and in the presence of vancomycin after 24 h of incubation. *Represents a statistically significant difference compared with S. aureus 0 h and S. aureus 24 h (p<0.05); B—S. aureus growth inhibition for each time point of released vancomycin. (2) No growth; (1) Growth in one replicate; (11) Growth in two replicates; (111) Growth in five replicates.

FIG. 6: A—S. aureus adhesion onto nanoHA, nanoHA/collagen, and heparinized nanoHA/collagen granules expressed as CFU per mm² of granules. *Represents a statistically significant difference compared with nanoHA granules (p<0.05); B—SEM images of adherent S. aureus on granules (1, nanoHA; 2, nanoHA/collagen; and 3, heparinized nanoHA/collagen).

FIG. 7: Metabolic activity of MC3T3-E1 cells cultured with heparinized nanoHA/collagen granules with and without vancomycin. Results are expressed in terms of relative fluorescence units (RFU) per mm² of granules. TCPS was used as a control. *Represents a statistically significant difference compared with heparinized nanoHA/collagen granules without vancomycin for the same time point (p<0.05). **Represents a statistically significant difference compared with heparinized nanoHA/collagen granules with vancomycin at 24 h and 4, 7, and 14 days (p<0.05). ***Represents a statistically significant difference compared with heparinized nanoHA/collagen granules without vancomycin at 24 h and 4 and 7 days (p<0.05).

FIG. 8: SEM images of MC3T3-E1 cells morphology on heparinized nanoHA/collagen granules with and without vancomycin after 1, 4, 7, 14, and 21 days of culture.

FIG. 9: CLSM images of MC3T3-E1 cells morphology on heparinized nanoHA/collagen granules with and without vancomycin after 1, 4, 7, 14, and 21 days of culture. F-actin is represented in red, whereas cell nuclei were counterstained in blue with Hoechst stain.

### DETAILED DESCRIPTION

The present solution will be described in detail. A composition/material with chemical and morphological similarities with the extracellular matrix of bone tissue, capable to prevent or treat osteomyelitis via a controlled release of vancomycin and subsequently induce regeneration of bone tissue that often undergoes necrosis due to infection, has been developed. Heparinized nanoHA/collagen porous granules were prepared and characterized.

The present solution reports the immobilization of heparin at NanoHA/collagen granules allowing a sustained release of antibiotics to be applied on the infected bone. For this, granules were loaded with antibiotic, in particular vancomycin, and its release profile was analysed, as well as its bioactivity against S. aureus bacteria. In terms of in vitro biological assays it was also investigated the adhesion of S. aureus to granules as well as cell viability pre-osteoblast MC3T3-E1 in the presence of vancomycin.

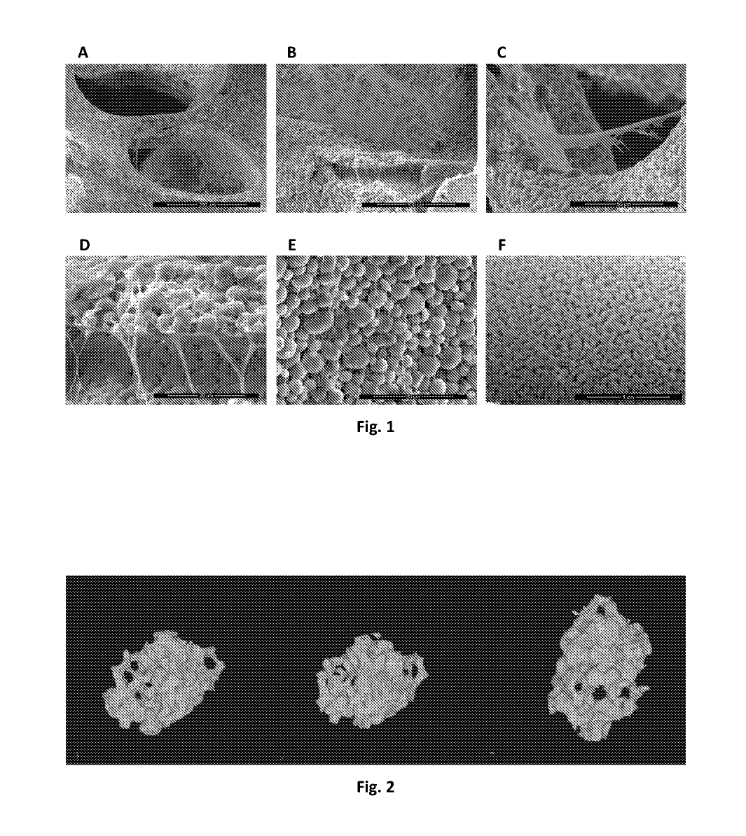

In an embodiment, NanoHA granules were obtained by crushing sintered scaffolds and at the end passing them in sieves with pore size between 1.18 and 1.70 mm, obtaining granules with a granulometry between those two values. Scaffolds were prepared using polyurethane sponge impregnation method. Briefly, polyurethane sponges (Recticel, Belgium) were impregnated with nanoHA slurry. The slurry was prepared, in particular, using a ratio of 5:4.4:0.2, respectively, of nanoHA powder (g), ultrapure water (mL), and dispersive agent Dolapix CE64 (mL) (Zschimmer & Schwarz, Germany). The nanoHA is a highly pure spray-dried powder with an average particle size of 5.0±1.0 μm nanoXIM.HAp202 (Fluidinova SA, Portugal), being composed by highly crystalline nanoparticles aggregates, as confirmed by high-resolution transmission electron microscopy (FIG. 1). The impregnated sponges were dried, in particular at 37° C. in the oven for approximately 30 min and then heat-treated in a sintering furnace (Thermolab). The heat treatment cycle used was as follows: heating rate of 1° C./min till 600° C. with 1 h plateau, followed by a heating rate of 4° C./min till 830° C. with 1 h plateau. Afterward, the samples were naturally cooled inside the furnace.

The collagen inclusion, crosslinking and heparin immobilization were carried out, in particular, as follows. A 0.5% (w/v) collagen solution was prepared by dissolving type I collagen (bovine Achilles tendon, Sigma-Aldrich, St. Louis, Mo.) overnight in HCl (0.01 M, pH=2) at 4° C. The solution was then homogenized for 3 h using an Ultra Turrax (T25 D, IKA®) at 10000 rpm on ice and then diluted to a 0.05% solution. The nanoHA granules were spread on petri dishes, and a single drop of collagen solution was applied in each granule. Finally, the nanoHA granules were placed in a vacuum oven (Binder, Germany) at room temperature (RT, 25° C.) for 48 h to allow collagen to penetrate the granules.

Collagen chemical crosslinking was performed using N-(3-dimethylaminopropyl)-N′-ethylcarbodiimide (Aldrich, St. Louis, Mo.) and N-hydroxysuccinimide (Fluka). Briefly, 27.6 mg of N-(3-dimethylaminopropyl)-N′-ethylcarbodiimide (EDC) and 10 mg of N-hydroxysuccinimide (NHS) were dissolved on 12 mL of 2-mofpholinoethane sulfonic acid (MES) buffer (0.05 M, pH=5.4, Sigma, St. Louis, Mo.). The reaction was carried out for 2 h at 4° C. After the crosslinking reaction, the solution was removed, and the samples were washed three times with MES buffer and dried overnight in a vacuum oven. Considering heparinized nanoHA/collagen granules, the collagen crosslinking and heparin immobilization were performed as described earlier, with the addition of heparin, along with immobilization stoppage using phosphate-buffered saline (PBS) and a washing procedure with NaCl (4 times for 6 hours) and ultrapure water (4 times for 8 hours) and dried overnight in a vacuum oven at room temperature.

Granules morphology, collagen distribution, and chemical characterization were studied using scanning electron microscopy (SEM) and energy-dispersive X-ray spectroscopy (FEI Quanta 400 FEG SEM/EDAX genesis X4M) with an acceleration voltage of 15 kV. Previously, the samples were fixed with Araldite on the aluminum sample holder and sputter coated with an Au/Pd alloy thin film for 90-110 s (SPI Module Sputter Coater) to yield them electrical conductivity.

Three-dimensional (3D) structure of nanoHA and nanoHA/collagen granules were assessed with X-ray micro-computed tomography (micro-CT) Skyscan 1072 scanner (SkyScan, Kontich, Belgium) in high-resolution mode of 6.69 mm x/y/z. Granules were scanned for approximately 1 h each using a pixel size of 3.29 mm. The energy and current of the X-ray source was 57 kV and 175 mA, respectively. A total of 250 slice images (two-dimensional) were considered and converted into binary images using a lower gray threshold of 60 and an upper gray threshold of 255, in order to distinguish ceramic material from pore voids. The slice images were assembled to yield 3D images and reveal quantitative morphological parameters. For two-dimensional and 3D image processing and visualization, two SkyScan software were used: CT Analyzer v.1.12.0.0 to obtain the morphological data and CTVox to create the 3D models of the granules.

The chemical composition of nanoHA, nanoHA/collagen, and heparinized nanoHA/collagen granules and crosslinked collagen was assessed with attenuated total reflectance Fourier transform infrared spectroscopy (ATR-FTIR). For that purpose, an FTIR spectrometer (Perkin Elmer 2000, Perkin Elmer, Waltham, Mass.) was used, with a resolution of 4 cm′ and a frequency region from 400 to 4000 cm′, and 100 scans were accumulated per sample. To perform ATR-FTIR, a Split Pea accessory (Harrick Scientific, Pleasantville, N.Y.) was used containing a silicon hemispherical crystal.

The vancomycin adsorption and release kinetics from granules were also performed. In particular, vancomycin loading was performed by immersing 20 mg of granules in Eppendorf tubes and in 1 mL of vancomycin solution (Vancomicina Combino Pharm) with a concentration of 25 mg/mL (pH=4). The antibiotic adsorption onto granules was performed at 37° C. and 120 rpm in the orbital shaker (KS 4000 IC, IKA®) for 24 h.

After loading, the supernatant solution was removed, and the granules were transferred to new Eppendorf tubes, and 1 mL of PBS (pH=7.4) was added. Eppendorf tubes were placed in the orbital shaker at 37° C. and 120 rpm. To determine the vancomycin release from the granules, 200 mL of solution was withdrawn and replaced by fresh PBS solution after 0.25, 0.5, 1.5, 2.5, 3.5, and 24 h and from then onward every 24 h up to 360 h. Control experiments using antibiotic-free ceramic samples was performed under the same experimental conditions (negative control). The removed solution was centrifuged for 5 min and 14000 rpm to avoid particles in suspension. Vancomycin concentration was determined by molecular absorption spectroscopy at 280 nm using a spectrophotometer (Lambda 35 UV/Vis Spectrometer, Perkin Elmer). The collected samples were subsequently frozen at −20° C. to per-form microbiology assays. All tests were performed in triplicate.

Vancomycin bioactivity was assessed using broth microdilution method. Therefore, S. aureus ATCC 25923 was grown on nutrient broth (Uofilchem, Italy) for 24 h at 37° C. and 120 rpm. From that bacterial suspension, an inoculum was taken and adjusted to an absorbance (640 nm) of 0.2, corresponding to 3.8×10⁸ colony forming units (CFU)/mL. Afterward, 96-well plates were filled with bacterial suspension (180 μL) and with the released vancomycin (20 μL). For each time point, eight wells were used. Nutrient broth without bacteria and bacterial suspension without vancomycin were established as controls. The plates were incubated for 24 h at 37° C. and 120 rpm. After incubation, the absorbance was measured at 640 nm using a microplate reader (Spectramax M2e, Molecular Devices, Sunnyvale, Calif.). The absorbance values were converted to total number of bacteria/mL using a calibration curve.

The adherence of S. aureus on the three type of granules was studied to see whether the bacterium has some tendency to migrate to one type of granules rather than to another. With that objective, a nutrient agar (Liofilchem, Italy) plate inoculated with S. aureus ATCC 25923 was used to create a 1.5×10⁸ CFU/mL suspension in 0.9% NaCl, which equals to 0.5 Mcfarland equivalence turbidity standard in ambient light, using a densitometer (BioMerieux, France). To allow bacterial adhesion to granules, 20 mg of granules were placed in a glass tube with 1 mL of bacterial suspension and incubated in a gently shaking water bath at 37° C. for 1 h. The experiment was performed in triplicate. After incubation, each sample was washed twice with 0.9% NaCl to remove loosely adhered or nonadherent bacteria. Then, 5 mL of 0.9% NaCl was added to each tube and sonicated for 1 s at 20 kHz using a sonicator (Sonoplus HD 2200, Bandelin, Germany) with a MS 73 probe. The sonicated solutions were used to obtain serial dilutions, and these were placed onto nutrient agar culture plates and incubated at 37° C. for 18 h. Afterward, the number of adherent bacteria was counted, and the number of CFU/mm² was determined.

S. aureus was fixed using 1.5% (v/v) glutaraldehyde in cacodylate buffer (0.14 M) for 10 min and dehydrated in graded series of ethanol solutions. The samples were dried overnight at RT. Adherent S. aureus on granules were visualized using SEM as previously described.

The pre-osteoblasts MC3T3-E1 cells, an osteoblastic cell line derived from mouse calvaria, were grown in alpha minimum essential medium (α-MEM, Gibco, Life Technologies, Grand Island, N.Y.), supplemented with 1% penicillinstreptomycin (Gibco) and 10% fetal bovine serum (FBS, Invitrogen, Carlsbad, Calif.). Cells were incubated in a humidified environment at 37° C. and 5% of CO₂. Vancomycin adsorption on granules was performed as mentioned earlier. Granules were placed in 96-well plates, and cells were seeded using a density of 5×10⁴ cells/mL. Cells were cultured for 21 days, and medium was changed three times a week. As control, cells were cultured on tissue culture polystyrene (TCPS) with the same conditions used for the granules. For each condition, six replicates were used for the resazurin assay.

In an embodiment, the metabolic activity was also performed. The nontoxic alamar blue (resazurin) dye was used to determine the metabolic activity of MC3T3-E1 cells. The blue nonfluorescent dye is metabolized by cells, converting it to a reduced pink fluorescence dye. Therefore, 10% (v/v) of resazurin was added to the medium and incubated for 4 h at 37° C. and 5% of CO₂. Afterward, 100 μL was transferred into a black 96-well plate, and fluorescence was measured at 530 nm excitation and 590 nm emission wavelength with a fluorescence reader (SynergyMix, BioTek, Winooski, Vt.) using Gen5 1.09 Data Analysis Software. These measurements were made at 24 h and 4, 7, 14, and 21 days of culture.

Granules with seeded cells were rinsed with PBS and fixer using 1.5% gluteraldehyde (v/v) (Agar) in 0.14 M sodium cacodyl-ate buffer (Merck, Kenilworth, N.J.) at RT for 30 min. The samples were washed twice with PBS and then dehydrated in graded series of ethanol. Hexamethyldisilazane (Sigma) was added, and the samples were dried overnight at RT. Granules were visualized on SEM as previously described.

Confocal laser scanning microscopy. Granules with seeded cells were fixed using 4% paraformaldehyde for 15 min and then washed twice with PBS. Triton X-100 0.1% was used, for 5 min, to permeabilize the cells, and the cells were then incubated in 1% bovine serum albumin at 37° C. for 30 min. After incubation, the staining of the F-actin filaments was performed using Alexa Fluor 594 Phalloidin (1:100, Molecular Probes A12379, Invitrogen) in 1% bovine serum albumin at RT for 20 min in dark. The samples were then washed twice with PBS. Cell nuclei were stained with a solution of Hoechst dye (1:1000, Sigma) in PBS at RT for 15 min in dark. Finally, the samples were washed twice with PBS, and one drop of Vectashield was added. The images were acquired with a Leica SP5 Confocal microscope, using a 320 oil immersion objective. The obtained images were processed with Leica Application Suite version 2.6.0.

After proper approval by Direcção-Geral de Alimentação e Veterinária (DGAV), the Portuguese National Authority for Animal Health, proceeded to an in vivo studies using animal models was conducted.

Twelve animals were submitted to the development of experimental osteomyelitis by intramedullary placement in the left tibia of a metal fragment previously dipped in a suspension of S. aureus methicillin-resistant. In all animals it was possible to replicate the model and develop osteomyelitis associated with implant confirmed by macroscopic and microbiological evaluation at the time of removal. In four of the twelve animals it was placed the composition/material and in other four animals it was placed the composition/material with vancomycin. Macroscopically it was possible to verify the healing of bone infection in animals of the second group, associated with an excellent integration of the graft at 8 weeks of implantation. The histological studies of surgical specimens are in course to confirm the infection healing and evaluation of bone integration of the composition/material.

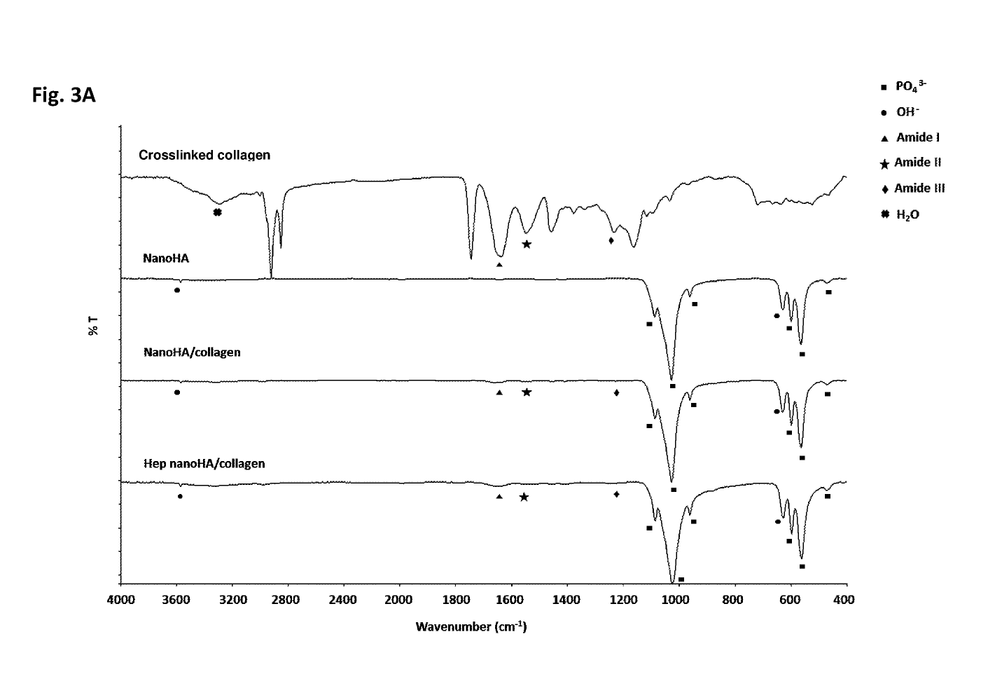

The produced granules were characterized by SEM analysis regarding morphology and collagen distribution. The obtained images revealed the presence of interconnective macroporosity (FIG. 1A). Moreover, SEM shows that collagen is distributed heterogeneously on nanoHA granules. For example, it can form large collagen fibres across a macropore (FIGS. 1B and 1C) or smaller fibres covering the nanoHA grains (FIG. D and 1E). The presence of microporosity (FIG. 2E) and nanoporosity (FIG. 1F) was also evident.

Micro-CT was performed to visualize the 3D structure of the granules and to determine porosity 62.7±1.5%, mean pore size (227±7 μm), and surface area (26.6±5.6 mm²). The images obtained for the 3D structure of a nanoHA granule show its irregular morphology and interconnective macroporosity (FIG. 2).

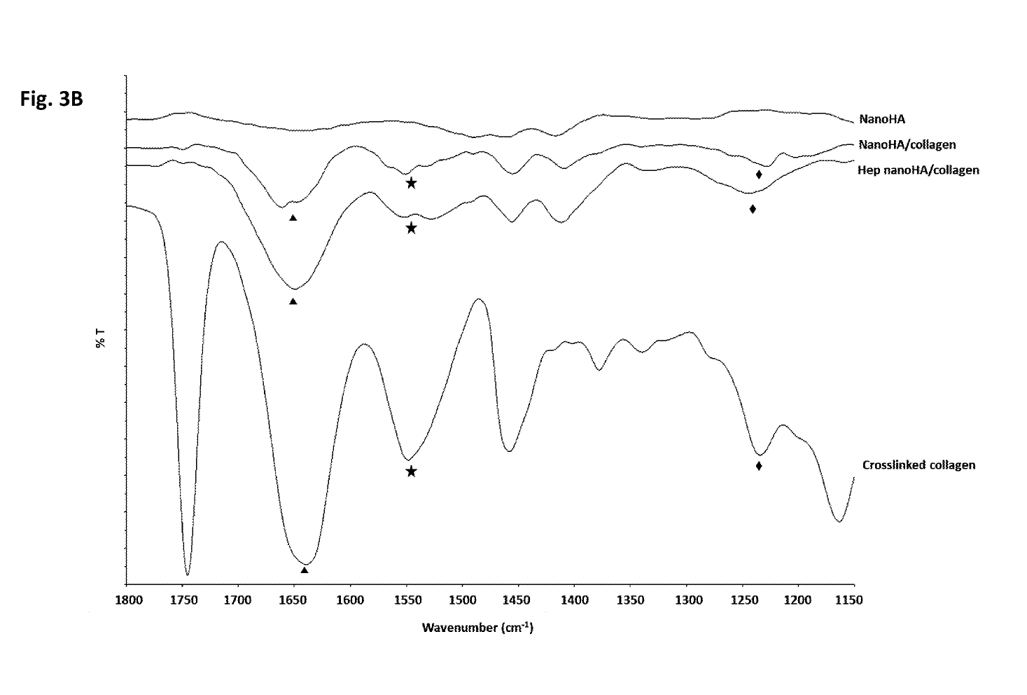

The chemical composition of the granules was assessed using ATR-FTIR (FIG. 3). The spectra revealed phosphate groups at 473, 565, 600, 962, 1028, and 1088 cm⁻¹. The bands at 630 and 3572 cm⁻¹ correspond to OH⁻ vibrational and stretching modes, respectively. Considering the samples containing collagen, the characteristic peaks for amide I (C═O stretching at 1600-1700 cm⁻¹), amide II (N—H deformation at 1500-1550 cm⁻¹), and amide III (N—H deformation at 1200-1300 cm⁻¹) were obtained. The crosslinked collagen sample has a broad band at 3200-3600 cm⁻¹, indicating the presence of adsorbed water on the material. For the heparinized nanoHA/collagen granules, there was no novel peaks present, as heparin peaks are superimposed over collagen ones.

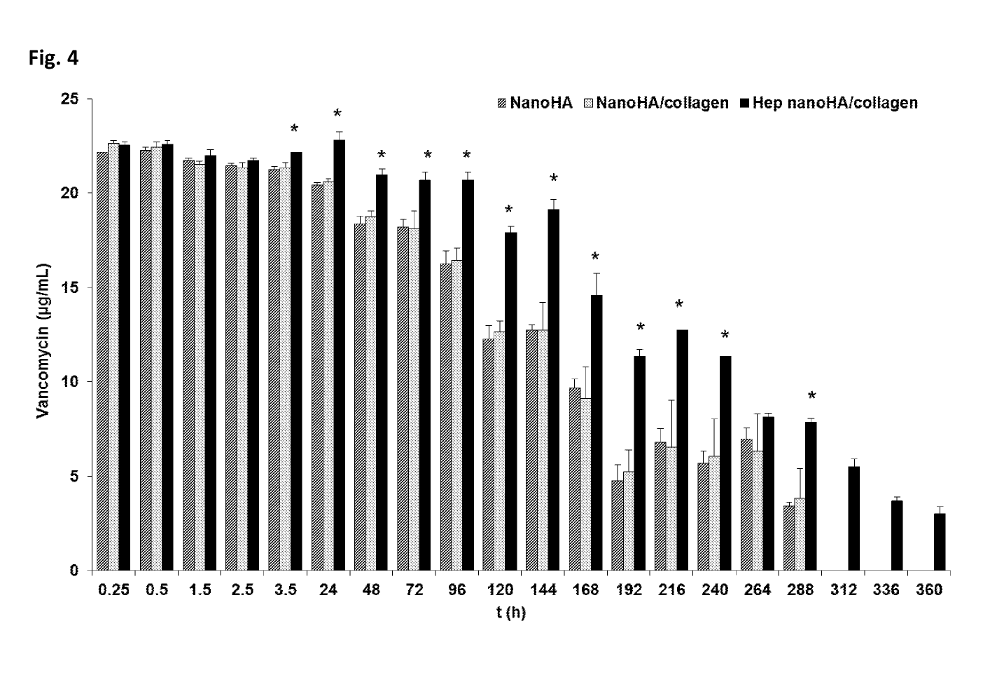

The vancomycin release from granules of nanoHA, nanoHA/collagen, and heparinized nanoHA/collagen and vancomycin bioactivity is shown in FIG. 4. For the three types of granules, an initial burst of antibiotic was observed in the first 15 min. The amount of vancomycin released in the first 24 h was equal for the three types of granules. However, after 24 h, the amount of released antibiotic decreased for the nanoHA and nanoHA/collagen granules. In opposition, the heparinized granules continued a more sustainable release, with higher amounts of vancomycin present, when compared with nonheparinized granules. After 12 days, there was no antibiotic detected for nanoHA and nanoHA collagen granules. For the heparinized granules, the vancomycin release stopped after 15 days. Considering the released concentrations detected, they were always higher than vancomycin minimum inhibitory concentration (MIC) for S. aureus ATCC 25923 (1 μg/mL) and below vancomycin minimum toxic concentration (MTC) (50 μg/mL) for the three types of granules.

In an embodiment, the present solution shows a therapeutic window in which the released concentrations are capable of inhibiting bacterial growth without toxicity being obtained. Moreover, a release profile, which starts with a sharp and continuous release of drug after more controlled manner, is considered ideal to ensure eradication of the bacteria. Also the final steps of releasing provide evidence that there is a fast decay of the available antibiotic ensuring that it will not remain being released below the MIC value, not to cause bacterial resistance.

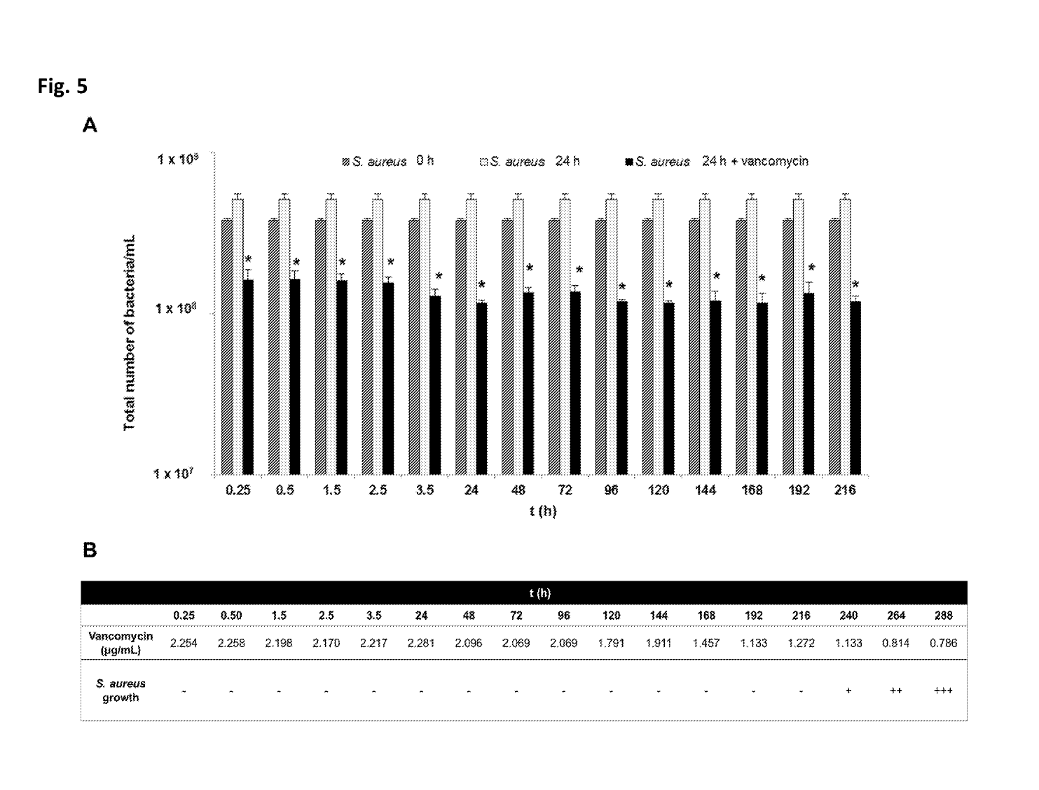

As heparinized granules presented the best release profile, the antibiotic bioactivity was assessed. The results show that the released antibiotic was able to inhibit S. aureus growth for 216 h (9 days) (FIG. 5A). After this time point, bacterial growth was observed in some replicates (FIG. 5B), and after 288 h, bacteria grew in all replicates. However, this was due to the method used to check the bioactivity (broth microdilution method), which requires a 10× dilution of the released initial concentration. Thus, the applied concentration in the assay is ten times lower, which means that the final concentrations are near or even below the MIC, and is therefore expected to occur bacterial growth and not as a limitation of the present disclosure. FIG. 5 indicates the concentration values of antibiotic applied in bacterial suspension. After 216 h, the concentrations were lower than the MIC, and it was expected that bacteria would grow. If not diluted, the release concentrations would be able to inhibit bacterial growth, because they were always higher than the MIC for S. aureus ATCC 25923.

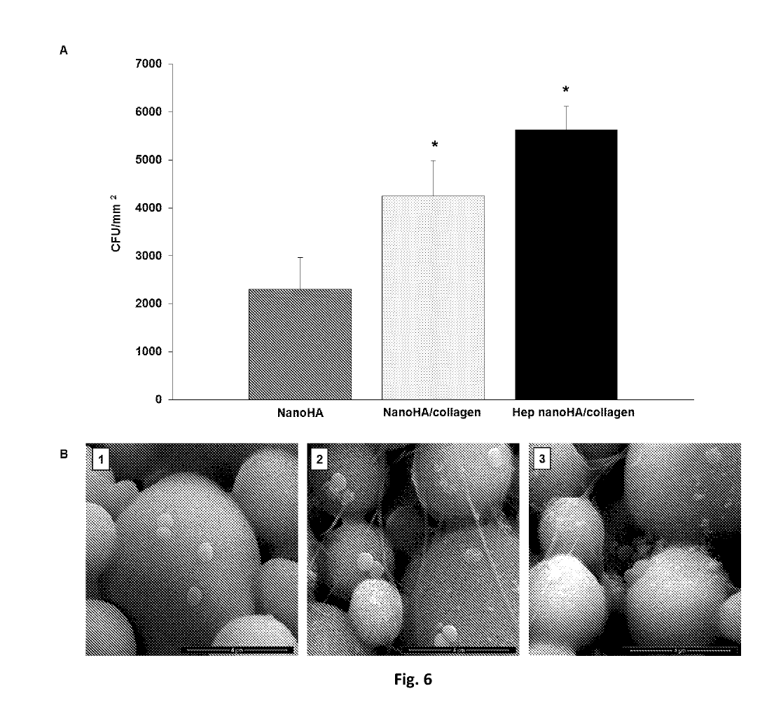

Bacterial adhesion assays were also performed with S. aureus to verify the interaction of the bacteria with the material. The bacterial adhesion was studied in granules of NanoHA, NanoHA/collagen and NanoHA/collagen heparinized. S. aureus adhesion on granules. FIG. 6 shows the results obtained for the bacterial adhesion studies. It was observed that granules containing collagen have higher number of adherent S. aureus, when compared with nanoHA granules (FIG. 6A). SEM images of adherent bacteria on granules surface show that bacteria adhered alone or in pairs. Moreover, bacteria were frequently seen near the collagen fibres and between the nanoHA grains (FIG. 6B). Since the bacteria have a natural tendency to migrate to the composition/material of the present subject matter, the existence of an antimicrobial agent will cause the bacteria to find more easily and are more effectively eradicated.

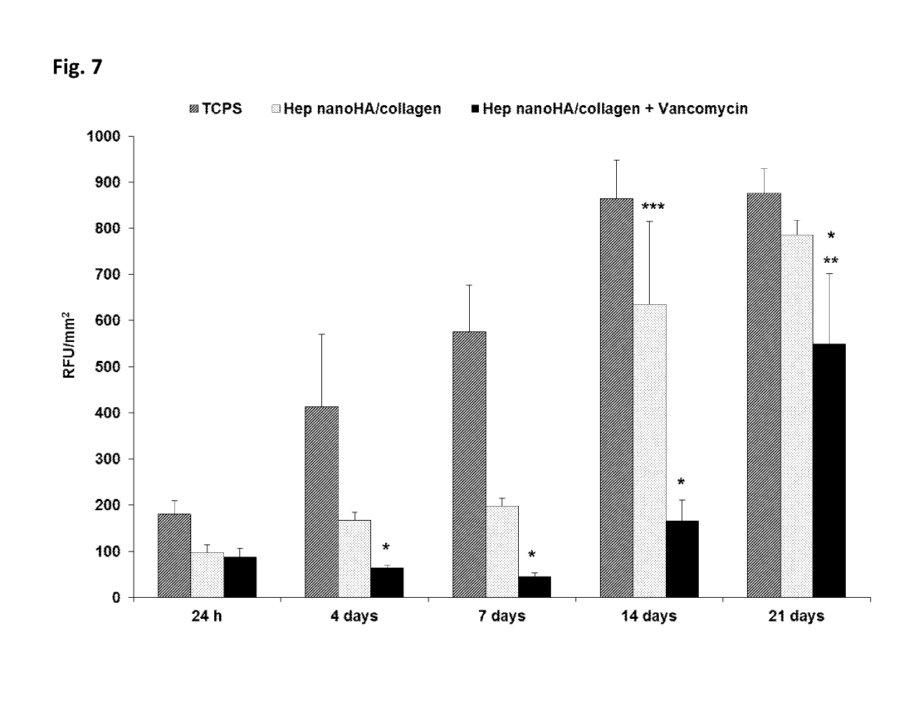

The effect of vancomycin release in pre-osteoblasts MC3T3-1 was analyzed. As NanoHA/heparinized collagen granules showed the best release profile of vancomycin, the effect of this antibiotic released in pre-osteoblasts was evaluated. The metabolic activity of cells and their morphology in the presence and absence of vancomycin, was studied. In FIG. 7, it is shown that there is no difference in the metabolic activity after 24 h of culture for cells with and without vancomycin. In the absence of this antibiotic, the metabolic activity of the cells increased continuously until the end of the culture. Considering the cells in the presence of vancomycin, a decrease in metabolic activity was observed until 7 days of culture. However, after 14 days of culture, it was observed that cells in the presence of vancomycin increased their metabolic activity and continued to increase it until the end of the culture (21 days), demonstrating the viability of the cells and their ability to recover in the presence of vancomycin. The effect of vancomycin release in pre-osteoblasts MC3T3-E1 was unexpected.

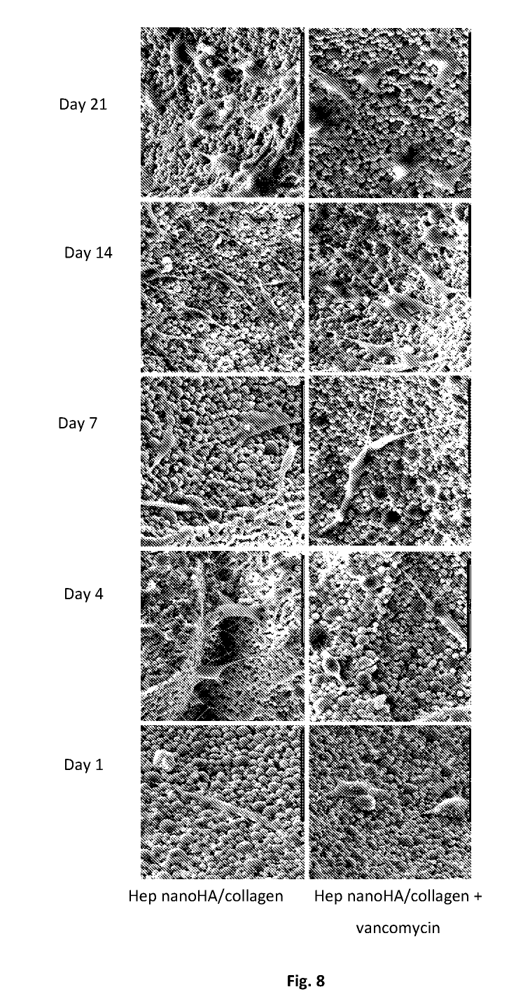

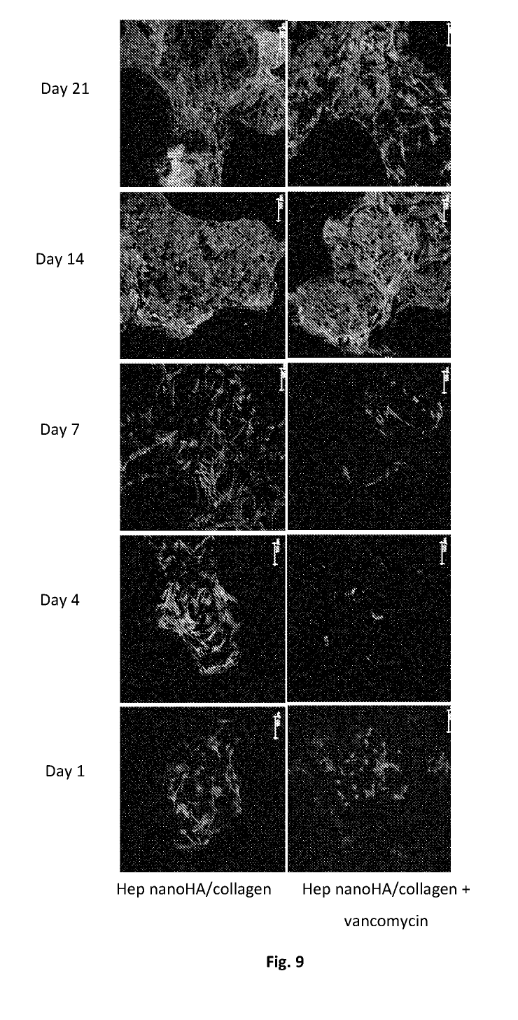

SEM images (FIG. 8) show that after 24 h, MC3T3-E1 cells without vancomycin are elongated and spread on the granules surface. In opposition, the MC3T3-E1 cells in the presence of antibiotic did not acquire an elongated shape after 24 h. Though, after 7 days, all cells were elongated and well spread on the granules surface, as it happened with cells without antibiotic. After 14 days, an increase in cell number was observed for both cells with and without antibiotic, indicating that cells were proliferating. The results obtained with confocal microscopy are consistent with those from SEM. For the first time points (24 h and 4 days), it was observed that MC3T3-E1 cells adhered on both granules, but the cells in the presence of vancomycin had a more rounded morphology (FIG. 9). After 7 days, it could be seen that cells in the presence of antibiotic started to elongate on the surface of the material. After 14 days of culture, both MC3T3-E1 cells with and without vancomycin are well spread, covering the entire surface.

The present solution relates to the production of an innovative 3D controlled releasing system aimed at improving the treatment of osteomyelitis through local antibiotic release. Porous heparinized nanoHA/collagen granules were successfully produced and exhibited interconnective macro-, micro-, and nanoporosity. This material also allowed a more sustainable and controlled release of vancomycin for 360 h (15 days), when compared with nonheparinized granules. The antibiotic released from heparinized granules was bioactive and capable of inhibiting S. aureus growth. The bacterial adhesion studies revealed that S. aureus adhere in higher number on granules containing collagen, and this behavior may improve their eradication. The presence of vancomycin did not affect the viability of MC3T3-E1 cells, as they were able to remain viable in the presence of the antibiotic and proliferate after 14 days of culture. The solution herein reported can first eradicate osteomyelitis and then promote the renewal of bone.

The present solution is not, obviously, in any way restricted to the herein described embodiments and a person with average knowledge in the area can predict many possibilities of modification of the same solution and substitutions of technical characteristics by others equivalent, depending on the requirements of each situation, as defined in the appended claims.

The embodiments described above can be combined with each other. The following claims further define the preferred embodiments of the present solution.

1. A pharmaceutical composition comprising
one or more granules containing calcium phosphate, collagen and one or more polymers of heparin, and
an antibiotic in an effective therapeutic amount,
wherein the antibiotic is bound to the heparin polymers.

2. The composition of claim 1, wherein the calcium phosphate granules are coated with the collagen.

3. The composition of claim 1, wherein the heparin polymers are bound to the collagen.

4. The composition of claim 1, previous claim wherein the antibiotic is a glycopeptide or penicillin or mixtures thereof.

5. The composition of claim 1, wherein the antibiotic is selected from the following list:
vancomycin, tobramycin, cefadroxil, cephamycin, cefazolin, cephalexin, cefaclor, cefoxitin, cefprozil, cefuroxime, cefdinir, cefixime, cefoperazone, cefotaxime, cefpodoxime, ceftazidime, ceftibuten, ceftozoxima, ceftriaxone, cefepime, rifampin, ofloxacin, fusidic acid, trimethoprim-sulfamethoxazole, penicillin or their mixtures.

6. The composition of claim 1, wherein the granules of calcium phosphate aggregates comprise dimensions between 0.01-10 mm, preferably 0.1-5 mm, more preferably between 0.5-1.5 mm.

7. The composition of claim 1, wherein the collagen is type I collagen.

8. The composition of claim 1, wherein the calcium phosphate granules are hydroxyapatite granules.

9. The composition of claim 1, wherein heparin has a molecular weight between 17 000-19 000 Da.

10. The composition of claim 2, wherein the coated granules further comprise a crosslinking agent.

11. The composition of claim 10, wherein the granules of calcium phosphate aggregates are crosslinked with type I collagen and are further coated with heparin.

12. The composition of claim 1 wherein the crosslink agent is selected from the following list: N-(3-dimethylaminopropyl)-N′-ethylcarbodiimide, N-hydroxysuccinimide, or their mixtures.

13. The composition of claim 1, for use in at least one of human medicine or veterinary, regeneration of bone and/or cartilaginous tissues, treatment or prevention of bone and/or cartilaginous infections, and prevention or treatment of osteomyelitis.

14. (canceled)

15. (canceled)

16. (canceled)

17. The composition of claim 1, wherein the composition is an injectable form.

18. A biomaterial comprising the composition of claim 1.

19. The biomaterial composition of claim 18, wherein the material is composed of granule comprising dimensions between 0.01-10 mm.

20. A method for making the composition of claim 1 or the biomaterial of claim 18, comprising the following steps:
impregnating at least one polyurethane sponge with a calcium phosphate slurry, in particular nanohydroxyapatite slurry, to obtain an impregnated sponges;
drying the impregnated sponges;
heat-treating the impregnated sponges in a sintering furnace until the sponge evaporates in order to obtain calcium phosphate material, preferably nanohydroxyapatite material;
breaking and sieving the obtained calcium phosphate material, in particular nanohydroxyapatite material, in granules with sizes between 0.8-2.0 mm, preferably 1.18-1.70 mm;
coating the granules, in particular by dipping or spraying, with a collagen solution;
adding a crosslinking agent to immobilize polymers of heparin on the granules surface;
and adding a suitable antibiotic to the granules, in particular by dipping or spraying in an antibiotic solution.

21. The method of claim 20, wherein the drying step is perform at 37° C. for 30 min.

22. The method of claim 20, wherein the heat-treating step comprises:
a heating rate step of 1° C./min till 600° C. with 1 h plateau;
a heating rate step of 4° C./min till 830° C. with 1 h plateau;
and a natural cooling step inside the furnace.

23. The method of claim 20, wherein the crosslinking step is carried out at 2-10° C. for 30 min-4 h, more preferably at 4° C. for 2 h.
